(19) United States
(12) Patent Application Publication
(10) Pub. No.: US 2010/0324637 A1
(43) Pub. Date: Dec. 23, 2010

(54) ELECTRODE PROBE FOR MEDICAL APPLICATION

(76) Inventors: Erik Trip, Uffelte (NL); Erhard Flach, Berlin (DE); Christian Schnittker, Berlin (DE); Marc Steffen Schurr, Berlin (DE)

Correspondence Address: Intellectual Property Dept.;Dewitt Ross & Stevens SC, 2 East Mifflin Street, Suite 600, Madison, WI, 53703-2865, US

(21) Appl. No.: 12/783,594

(22) Filed: May 20, 2010

Related U.S. Application Data
(60) Provisional application No. 61/219,434, filed on Jun. 23, 2009.

(51) Int. Cl. A61N 1/05; A61M 5/00

(52) U.S. Cl. 607/116; 604/20

(57) ABSTRACT

An electrode probe for medical applications includes a tubular, flexible probe body, an electrode mounted at the distal end of the probe body, and an electrical supply line within the probe body and extending to the electrode. At least a portion of the distal end section of the probe body can be transformed into a radially broadened collar, preferably owing to relative motion between the electrical supply line and the probe body. Where a screw-in electrode is used, the distal end section may be deformed into a radially broadened collar when the electrode is screwed into the body tissue, owing to forces exerted by the body tissue onto the distal end section, and/or owing to forces exerted by the screw-in electrode on the distal end section via a thrust bearing.

### CROSS-REFERENCE TO RELATED APPLICATIONS

This application claims priority under 35 USC §119(e) to U.S. Provisional Patent Application 61/219,434 filed 23 Jun. 2009, the entirety of which is incorporated by reference herein.

### FIELD OF THE INVENTION

The invention relates generally to medical probes, with a preferred version of the invention relating more specifically to an implantable electrode probe with a tubular, flexible probe body, an electrode mounted at the distal end of the probe body, and an electrical supply line guided in the probe body to the electrode.

### BACKGROUND OF THE INVENTION

Electrode probes are routinely used, for example, as coronary pacemaker electrodes. They are variously described in patent literature, with examples being the patent publications DE 10 2005 039 040 A1, DE 198 00 697 A1, DE 20 2006 020 517 U1 and EP 88730200.8.

Probes that can be implanted in the body, especially electrode probes, should have a probe diameter as small as possible to ensure comfortable passage through the insertion site and blood vessels up to the desired implant site, and to better enable the use of small insertion tools. However, probes with small diameter increase the danger of causing unwanted perforation of body tissue at the implant site.

As known in the field, active ingredients can be administered by the probe, particularly an electrode probe, at the implant site. For example, in U.S. Pat. No. 5,571,163 A and U.S. Pat. No. 5,324,324 A1 it is proposed that the electrode tip be coated with anti-inflammatory medications in order to locally counteract the tissue irritation caused by the probe. In the case of electrode probes with a relatively small probe diameter, the small probe surface disadvantageously limits the area available for administering the active ingredient. Additionally, if the same amount of active ingredient is to be used on a smaller probe diameter, the reservoir of active ingredient extends over a larger length of the probe so that portions of the active ingredient are released at a relatively far distance from the implant site, which can reduce the therapeutic utility of the active ingredient.

Therefore, there is a desire for a solution to the competing objectives of obtaining an optimal electrode probe diameter for ease of implantability of the probe, and a suitable configuration for local discharge of active ingredient by the probe.

### SUMMARY OF THE INVENTION

An objective of the invention is to provide an implantable electrode probe that has a small probe diameter in order to better ensure comfortable implantation, but also offers high safety against perforation, and also makes efficient discharge of active ingredient possible at the implant site, i.e. at locations at which the probe can irritate tissue. The invention involves an electrode probe for medical applications—e.g., a coronary pacemaker electrode, an electrode for nerve stimulation, an electrode for the measurement of brain potential, or the like—includes a tubular, flexible probe body; an electrode mounted at the distal end of the probe body (for example, a tip electrode); and an electrical supply line that is guided in the body of the probe. One or more electrical contacts can be mounted along the probe body. At least a part of the distal end section of the probe body—preferably at a part directly next to the electrode—can be transformed by a displacement mechanism between a first radially narrower condition and a second radially broader condition. In the second condition, the distal end section of the probe body is radially broadened in relation to the remainder of the probe body and the electrode.

The electrode probe can thus be implanted with a thin distal end section, allowing more comfortable passage through the insertion gateway and blood vessels owing to the smaller radial dimension of the distal end section of the probe body. On the other hand, the distal end section of the probe body can be radially broadened at the implant site, thereby deterring tissue perforation and also providing a relatively large surface for local discharge of an active ingredient at the implant site. In addition, because of the spatial proximity (or contact) of the broadened area at the distal end section to the implant site, the active ingredient can be discharged directly at the site of the tissue irritation, achieving high therapeutic efficiency. Moreover, compared to a distal end section of the probe body that is not broadened, a greater amount of active ingredient can be provided at or close to the implant site.

Throughout this document, the term “axial” will generally be used to describe directions along the length of the electrode body (inner tube), and the term “radial” will generally be used to describe directions perpendicular to the length of the electrode body.

In a preferred version of the electrode probe, the electrode mounted at the distal end of the probe body is mechanically coupled at least in an axial direction with the distal end section of the probe body, for example, by being connected with and/or engaged behind the distal end section, and the electrical supply line to the electrode is displaceable in an axial direction relative to the probe body. The distal end section of the probe body is designed in such a way that at least a part of the distal end section can be deformed into a radially broadened collar by a force that is transmitted by the electrode to the distal end section (for example, by manually effecting relative motion between the electrical supply line and the probe body). The distal end section is resiliently flexible and/or elastic to effect the deformation into the broadened state. Thus, the electrode probe can be implanted at the implant site with a non-broadened distal end section, and by then generating relative motion between the electric supply line and the probe body, the distal end section can be radially broadened.

In an advantageous version of the electrode probe, a casing surrounds at least a portion of the probe body, and is mechanically coupled to the distal end section in such a way that the distal end section can be deformed into the radially broadened collar as a result of relative motion between the electrical supply line and the casing (as by pushing the collar forwardly with respect to the supply line, and/or by pulling the supply line rearwardly with respect to the casing).

The electrode may be designed as a screw-in electrode for screwing into body tissue, for example, heart tissue, whereby rotation of the electrical supply line can displace the electrode between a passive setting within the probe body and an active setting at least partially outside of the probe body. Screw-in anchoring of an electrode is known, for example, from DE 20 2006 020 517 U1. At least a portion of the distal end section of the probe body is deformable into a radially broadened collar upon screwing the electrode into body tissue as the result of forces exerted on the deformable distal end section. Thus, the collar is formed in the distal end section of the probe body during screwing in of the electrode. The electrode probe can be positioned at the implant site with a non-broadened distal end section, and the distal end section can be radially broadened by screwing the electrode into the body tissue.

A screw-in electrode as described above can be displaced between the passive setting and the active setting by cooperating with a thrust bearing (“pitch provider”) mounted in the area of the distal end section at the probe body, during rotation of the supply line. Such a pitch provider is described in the aforementioned DE 20 2006 020 517 U1. The distal end section is designed in such a way that it can be deformed into a radially broadened collar by interaction between the screw-in electrode and the thrust bearing upon rotation of the supply line. The collar can thereby be easily and automatically formed in the distal end section of the probe body by rotating the supply line, with the collar being formed by the pressure exerted by the body tissue on the distal end section, and by the force transmitted by the thrust bearing to the distal end section. The electrode probe can therefore be positioned at the implant site with a non-broadened distal end section which is then radially broadened during anchoring by screwing the electrode into the body tissue, and by the transmission of force to the thrust bearing.

In an advantageous version of the electrode probe, at least a portion of the distal end section of the probe body bears through-holes which define lamellae (e.g., strips or fingers) extending in the axial direction, and which deploy into a radially broadened state. This can achieve simple formation of the collar with a relatively large radial broadened area.

In another advantageous version of the electrode probe, the distal end section of the probe body has several broadening components on its outer surface that can be displaced between a passive setting in which they abut the outer surface at the distal end section, and an active setting in which they extend from the distal end section in the radial direction. The broadening components may be coupled with a mechanical pre-loading tool, for example, a spring tool, that pre-loads the components into their active setting. A sleeve-shaped insertion tool can be used to force the broadening elements into their passive position, and after implantation, the insertion tool can be removed from the implanted electrode probe to move the broadening elements into their active setting.

The outer surface of the distal end section of the probe body can be provided with at least one active ingredient to be discharged into the body tissue, as by providing the active ingredient as a coating of the outer surface of the distal end section. This can achieve targeted local discharge of active ingredient near or in contact with the body tissue at the implantation site, whereby the therapeutic effectiveness of the discharged active ingredient is improved.

The distal end section of the probe body may include a discharge device connected with at least one active ingredient reservoir for discharging at least one active ingredient into the body tissue. This can also achieve targeted local discharge of active ingredient near or in contact with the body tissue at the implant site.

Alternatively or additionally, the electrode may be provided with a discharge device connected with at least one active ingredient reservoir for discharging at least one active ingredient into the body tissue at the implantation site. This can also provide targeted local discharge of active ingredient near or rather in contact with the body tissue at the implant site to enhance the therapeutic effectiveness of the discharged active ingredient. The active ingredient reservoir(s) can be compressed by a displaceable piston or other tool in order to effect transport of the active ingredient from the active ingredient reservoir to the discharge device. The compression means for compressing the active ingredient reservoir may be displaceable by coupling a swellable material to the compression means. A wall of the compartment for housing the swellable material may have one or more permeable openings for admitting liquid for swelling the swellable material. The available area of the liquid-admitting openings may be varied as the compression means displaces, such that the rate of swelling (and thus the motion of the compression means) may be varied with the position of the compression means.

Where a reservoir of active ingredient is provided, it is useful if the reservoir is housed in the probe body.

If a discharge device for discharging at least one active ingredient is used, it can be useful if the reservoir of active ingredient is in fluid connection with an electrically operated pump for supplying the active ingredient to the active ingredient discharge device.

### DESCRIPTION OF THE DRAWINGS

Exemplary versions of the invention are now explained in detail with reference to the accompanying drawings, wherein the same or functionally similar elements are labeled with the same legend. Shown are:

FIG. 1A-1B schematic perspective views of an exemplary first version of the electrode probe;

FIG. 2A-2C schematic perspective views of a variation of the first exemplary version of the electrode probe;

FIG. 3A-3D schematic views of a second exemplary version of the electrode probe;

FIG. 4A-4C schematic views of a third exemplary version of the electrode probe;

FIG. 5A-5D schematic views of a variation of the second or third exemplary version of an electrode probe;

FIG. 6A-6F schematic views of variants of the electrode tip for use in further designs of the electrode probe.

### DETAILED DESCRIPTION OF THE INVENTION

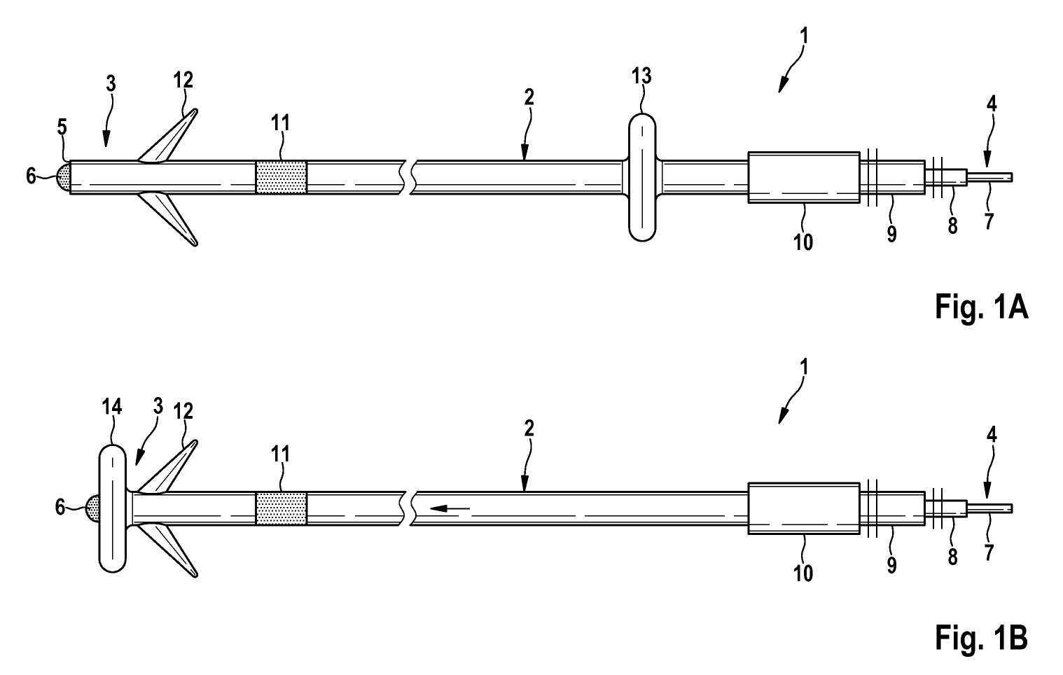

A first exemplary version of the electrode probe is schematically illustrated in FIGS. 1A and 1B. An electrode probe 1 designed, for example, for use as a heart pacemaker electrode, includes a tubular bendable probe body 2 with a distal end section 3 and a proximal end section 4, wherein the latter may include a standard connection (not shown) for connecting electrode probe 1 with a heart pacemaker (also not shown). The axial direction of the probe body 2 is defined by its direction of extension (its length), whereas the radial direction of the probe body is oriented perpendicular to its length.

At a facing surface 5 or otherwise on the distal end section 3 of the probe body 2, an at least substantially half-spherical tip electrode 6 is engaged radially about the distal end of the probe body 2, so that a force can be transmitted in the axial direction between the tip electrode 6 and the distal end 3 of the probe body 2. The probe body 2 forms a lumen housing an interior conductor 7, and a first isolation sleeve 8 made of an insulating material surrounds the conductor 7, wherein the conductor 7 and sleeve 8 can jointly be considered to be the “inner part” of the probe body 2. The interior conductor 7, which may be provided as a wire helix or in other forms, makes contact with the tip electrode 6 as an electrical supply line.

An exterior conductor 9, which can also be designed (for example) in the form of a wire helix, is surrounded by an isolation sleeve 10 (only partially shown) and extends as an electrical supply line to make contact with a ring electrode 11 located proximal to the distal end section 3. As illustrated in FIG. 1A, for example, the exterior conductor 9 can be spiraled onto a block 13 that lies between the distal end 3 and the proximal end 4 of the probe body 2. The exterior conductor 9 and the second isolation sleeve 10 can jointly be considered to be the “exterior part” of the probe body 2.

The interior part of the probe body 2 (the conductor 7 and/or sleeve 8, as defined above) is displaceable relative to the exterior part of the probe body 2 (the exterior conductor 9 and/or the second isolation sleeve 10) in an area between the distal end section 3 and the proximal end section 4. At the distal end section 3 of the probe body 2, the tip electrode 6 is connected with the probe body 2. At the area of the proximal section 4, the interior part of the probe body 2 may be connected to the exterior part of the probe body 2.

In the area of the distal end section 3, a number of anchor elements 12 are distributed circumferentially about the exterior surface of the probe body 2. These anchor elements 12 may take the form of tips (“tines”), and may be oriented at an angle of, for example, approximately 30° to the proximal end section 4 of the probe body 2. The anchoring elements 12 may serve in a known manner for so-called passive anchoring of the electrode probe 1 in the ventricular trabecular meshwork.

As is shown in FIG. 1B, and as indicated by an arrow in the axial direction, the distal end section 3 of the probe body 2 can be deformed by displacing the exterior part (exterior conductor 9 and/or the second isolation sleeve 10) in a distal direction relative to the interior part of the probe body 2 (the conductor 7 and/or sleeve 8) into a circular collar 14, which radially broadens the probe body 2 and tip electrode 6. The collar 14 is directly next to tip electrode 6. The distal end section 3 of the probe body 2 is designed to be correspondingly resilient for this purpose.

The exterior surface of the distal end section 3 of the probe body 2 is preferably coated with at least one pharmaceutically active substance (active ingredient), which can be discharged to the surrounding coronary tissue. The active ingredient might only be applied to that part of collar 14 that is aligned toward the coronary tissue. For purposes of dispensing the active ingredient, the distal end section 3 is preferably formed of (for example) a biocompatible polymer such as (again for example) silicone rubber and polyurethane, or of a material that can be resorbed and which releases the active ingredient on a delayed basis during decomposition. In principle, the distal end section 3 can be coated with any active ingredient, with preferred ingredients being anti-inflammatory steroids (glucocorticoids), especially alclomethason, amcinonide, beclomethasone, betamethasone, budenoside, ciclesonide, clobetasol, clobetasone, clocortolone, cloprednol, cortisol, cortisone, deflazacort, desonide, desoximethasone, dexamethasone, diflorasone, diflucortolone, fludroxycortide, flumetasone, flunisolide, fluocinolon, fluocinonide, fluocortin, fluocortolone, fluorometholone, fluprednides, fluticasone, halcinonide, halometasone, hydrocortisone, medrysone, methylprednisolone, mometasone, prednicarbate, prednisolone, prednisone, prednylides, rimexolone, tixocortol, triamcinolone, as well as derivatives thereof. Derivatization can be performed especially as aceponate, acetate, acetate-propionate, acetonide, benzoate, buteprate, butyrate, butyrate-propionate, diacetate, dihydrogenphosphate, dipropionate, ethyl carbonate propionate, hydrogensuccinate, hexacetonide, isonicotinate, palmitate, phosphate, pivalate, propionate, sodiumphosphate and valerate. The active ingredient can be dissolved or suspended in a matrix. A possible dose is, for example, in the range of 0.01 to 1,000 mg.

The electrode probe 1 schematically shown in FIGS. 1A and 1B can be implanted in straightforward fashion with a “thin”, i.e. un-broadened distal end section 3, of the probe body 2. After implantation, deformation can take place, for example, by manual displacement of the exterior part of the distal end section 3 in a distal direction relative to the interior part, thereby deforming the distal end section 3 to define the ring-shaped collar 14, with the radial broadening providing effective protection against perforation of the coronary tissue. As a result of the circular collar 14, at least one active ingredient, particularly a steroid, can be discharged near to or in contact with the coronary tissue, whereby high therapeutic effectiveness of the applied dosage can be achieved.

It is also possible to apply a cladding to the probe body 2 that is connected with the distal end section 3 of the probe body 2, whereby displacement of the distal end section 3 in a proximal direction forms the circular collar 14 relative to the interior part. Fixation of the collar 14 into its expanded form could be accomplished by affixing the interior and exterior parts of the probe body 2 together, as by use of a crimping/fixation sleeve (not shown) to which electrode probe 1 is mounted near the pacemaker pocket.

It would also be possible to create a spring tool consisting of, for example, nitinol in the distal end section 3 of the collar 14, whereby the electrode probe 1 is implanted with an insertion tool which is removed after the implantation so that the collar 14 can expand subject to the effect of the spring tool.

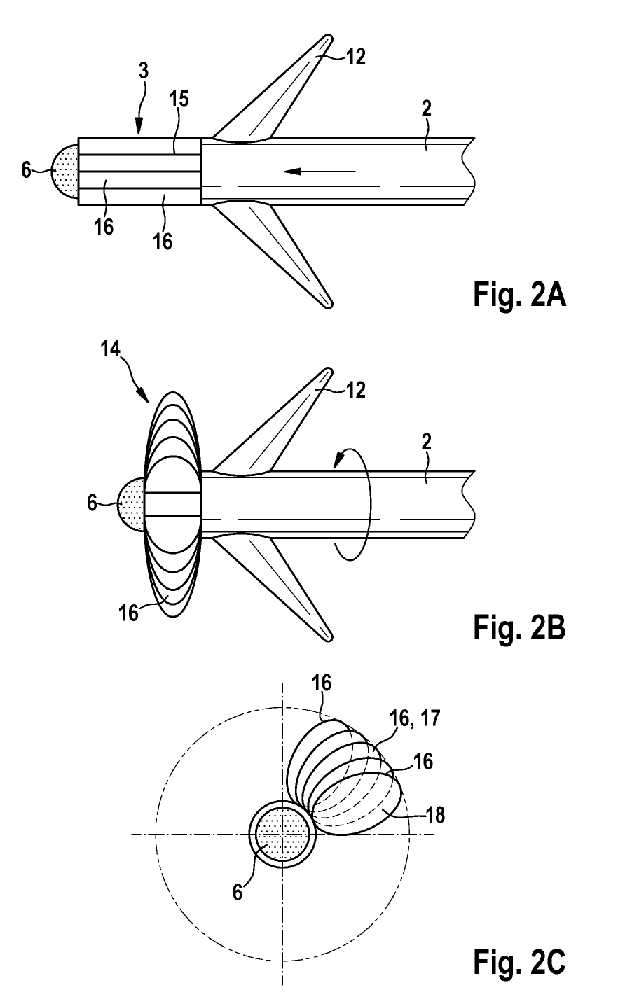

FIGS. 2A to 2C respectively show schematic perspective views of a variant of the first exemplary version of the electrode probe 1. In order to avoid unnecessary repetition, only the differences with the exemplary version shown in FIGS. 1A and 1B are now discussed. FIGS. 2A and 2B show a perspective lateral view of the distal part of the probe body 2, and FIG. 2C is a view of the same from the front.

The distal end section 3 of the probe body 2 is provided with a number of axial strips or lamellae 16 distributed in the circumferential direction. To form the lamellae 16, slots 15 are defined along the distal section 3. If the exterior piece is then displaced in a distal direction relative to the interior piece, the lamellae 16 then protrude to form the collar 14. Based on the lamellar structure of the distal end section 3, the collar 14 can be generated via application of a relatively small force, and the lamellae 16 can be deformed into a relatively broad collar 14 in the radial direction. As indicated by the arrow in FIG. 2B, by rotating the probe body 2 in a circumferential direction, it can also be achieved that not only the exterior surfaces 17 of the lamellae 16 of the collar 14 but also the interior surfaces 18 come in contact with the coronary tissue. If the interior surfaces 18 of the lamellae 16 are coated with an active ingredient, the surface of the collar 14 that is used for discharging active ingredient to the myocardium can thereby be enlarged in order to improve the therapeutic efficiency of the active ingredient.

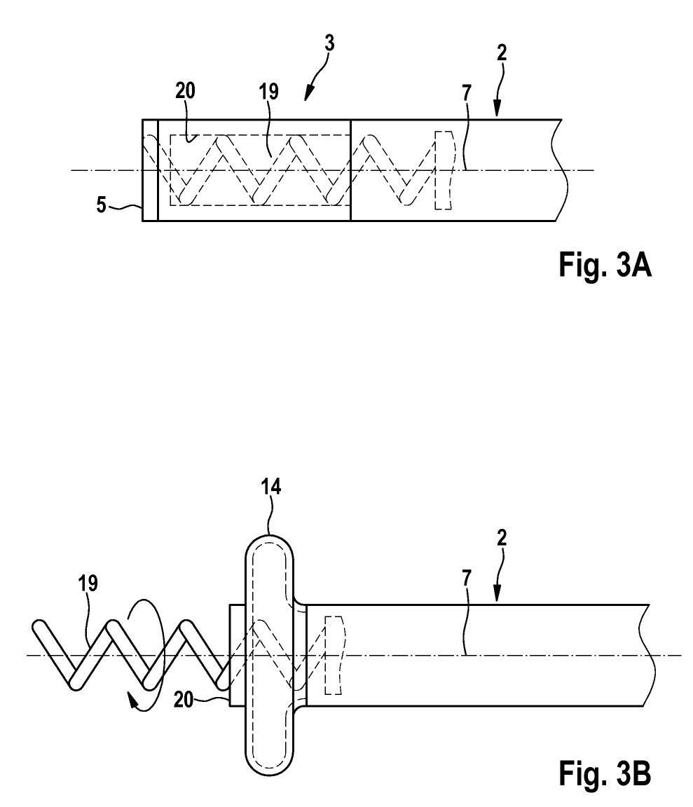

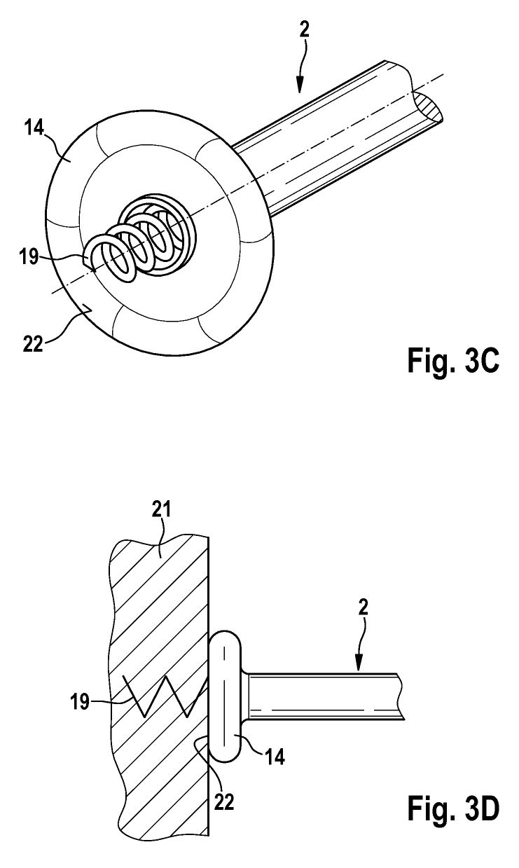

FIGS. 3A to 3D schematically illustrate the distal part of the second exemplary version of the electrode, with FIG. 3A showing an axial cross section view, and FIGS. 3B-3D respectively showing perspective views of the distal part of the probe body 2.

In FIGS. 3A to 3D, the electrode mounted at the distal end of the probe body 2 is designed in the form of a corkscrew-like (i.e. helical) electrode 19 for screwing into the myocardium 21. The screw-in electrode 19 is rotatably mounted in the lumen of the probe body 2 and connected with interior conductor 7, which has sufficient torsion stiffness that it may transmit torsion to the screw-in electrode 19 for screwing into the myocardium 21. The screw-in electrode 19 acts jointly with a thrust bearing 20 (pitch-provider), which is mounted on an interior side of distal end section 3 of the probe body 2. If the interior conductor 7 is rotated, the screw-in electrode 19 can be displaced between a passive position shown in FIG. 3A, in which it is located within the probe body 2, and an active position shown in FIG. 3B, in which it is in part positioned outside of the probe body 2. When screw-in electrode 19 is rotated out of the probe body 2 it exerts a force in the axial direction toward the proximal end 4 of the probe body 2. The mechanism for activating the screw-in electrode 19 is known in the field, for example, from DE 20 2006 020 517 U1.

If the electrode probe 1 is anchored at the implant site by screwing in electrode 19 into coronary tissue 21, the tissue exerts a proximally-oriented counter-force upon the distal facing surface 5 of the distal end section 3 of the probe body 2. Likewise, upon rotating screw-in electrode 19 out of the probe body 2, the thrust bearing 20 exerts a proximally-oriented force on the distal end section 3. Both effects contribute to deformation of the distal end section 3 into a radially broadened collar 14 during the screwing in of screw-in electrode 19, as illustrated in FIGS. 3B-3D. FIG. 3D shows electrode probe 1 anchored in coronary tissue 21, with the collar 14 having a two-dimensional area abutting the coronary tissue 21. The distal end section 3 is provided with sufficient deformability to make formation of a collar possible.

The electrode probe 1 illustrated in FIGS. 3A and 3D can thus be implanted in a comfortable manner with a cylindrical distal end section 3 of the probe body 2, and with the screw-in electrode 19 being housed in the lumen of the probe body 2 (see FIG. 3A). When the electrode 19 is screwed in at the implant site to anchor the electrode probe 1, the distal end section 3 of the probe body 2 radially enlarges as it axially shortens, forming circular collar 14 that radially broadens probe body 2. The face of the collar 14 abuts the surface of the coronary tissue 21 (see FIG. 3D). The collar 14 thus provides perforation protection, deterring perforation of the coronary tissue 21 by electrode probe 1.

By coating an exterior surface 22 (FIGS. 3C-3D) of the collar 14 that faces the coronary tissue 21 with an active ingredient (for example, a steroid), the active ingredient can be discharged to the site of tissue irritation by being targeted locally to the area 22 in direct contact with the coronary tissue 21, so that high therapeutic efficiency can be achieved.

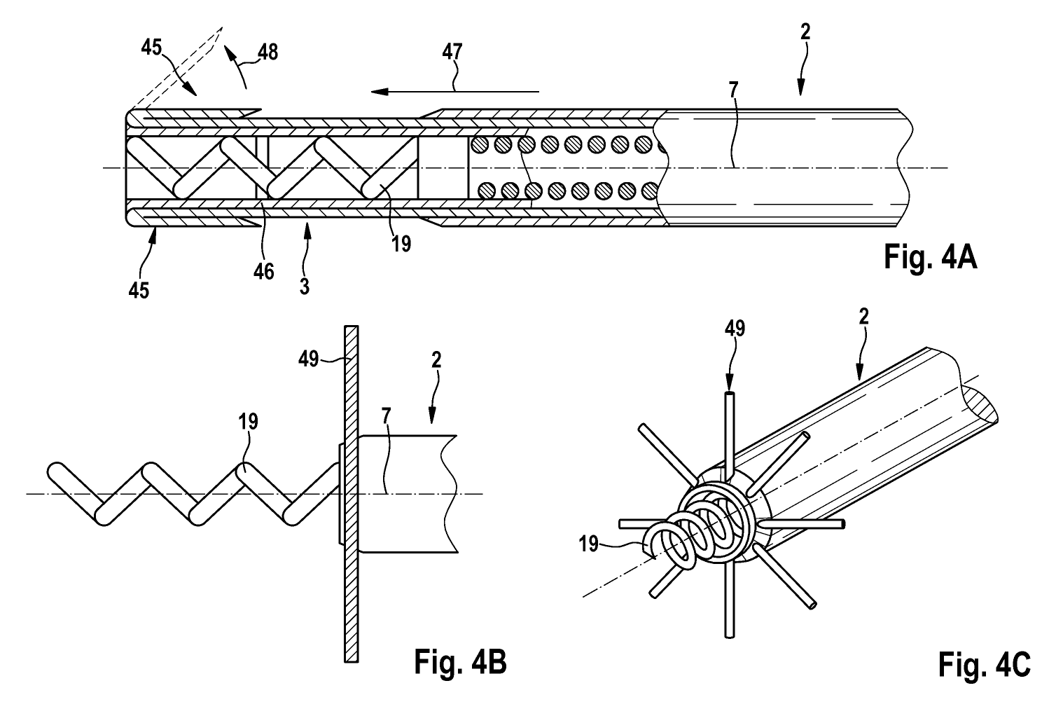

FIGS. 4A to 4C show the distal part of a third exemplary version of the electrode probe, wherein FIGS. 4A and 4B respectively show an axial cross section and FIG. 4C shows a perspective view of the distal part of electrode probe 1.

In FIG. 4A, at the tubular distal end section 3 of the probe body 2, extensions defined as finger-like lamellae 45 are radially distributed about the probe and hingedly mounted at the outermost tip of the probe, and abut the probe tip along their lengths. As a result of screwing in the electrode 19 at the implantation site, a sleeve 46 (pitch-provider) slides in the direction of arrow 47 under the lamellar extensions 45, whereby the extensions 45 swing radially upright in the direction of arrow 48. The radially upright extensions—shown at 49 in FIGS. 4B and 4C—abut the coronary tissue after installation of the probe. The sleeve 46 which slides under extensions 45 to deploy them can be shaped distally like a cone to ease slipping the sleeve 46 under the lamellae 45. The exteriors of the extensions 45 can be coated with an active ingredient.

Alternatively, by displacing the exterior part over the interior part (as discussed above with respect to FIGS. 1A and B), a sleeve 46 which is firmly connected with the displaceable exterior piece can be slid under the lamellae 45, whereby these are put upright in the radial direction.

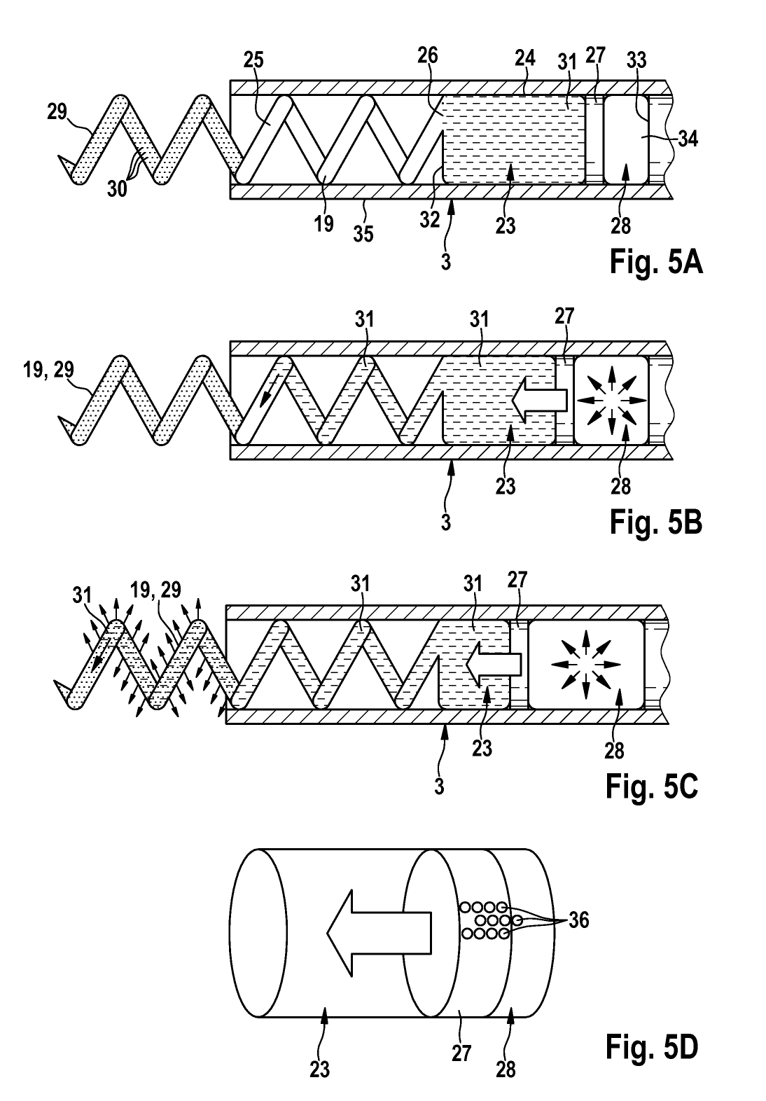

FIGS. 5A to 5D schematically illustrate another feature which may be incorporated in the foregoing probes. Here not only collar 14 (which is not shown for sake of simplicity), but also the screw-in electrode 19, may discharge at least one active ingredient. The electrode 19 has an internal passageway 25 connected to an opening 26 of a reservoir 23. A distal section 29 of the electrode 19 serves to anchor the electrode probe 1 in coronary tissue 21, and it bears pores 30 that open into the passageway 25. An active ingredient can be discharged through these pores 30 to the surrounding area, particularly when the electrode probe 1 is anchored to the coronary tissue 21.

The active ingredient reservoir 23 is defined by the housing wall 24, a piston 27, and an opposing distal facing surface 32. An active ingredient 31—for example, a steroid—can be discharged from the reservoir 23 through the pores 30 in the screw-in electrode 19. The piston 27 can be displaced within the housing wall 24 in the axial direction, and can be bounded on its side opposite the reservoir 23 with a swellable material 34 (FIG. 5A) such as cellulose, alginate, starch, or their derivatives or similar materials. The swellable material 34 is housed in a compartment 28 formed within the housing wall 24 adjacent the piston 27. The active ingredient reservoir 23 and the compartment 28 for the swellable material 34 are housed in the lumen of the probe body 2.

As illustrated in FIGS. 5B and 5C, the swellable material 34 can be made to swell upon contact with a liquid, e.g., blood, to displace the piston 27 in the axial direction. As a result, the active ingredient reservoir 23 is compressed and the active ingredient 31 is pressed through the pores 30 for discharge from the screw-in electrode 19. The housing wall 24, or rather the outer casing 35 of the probe body 2, may be provided with a number of openings 36 (FIG. 5D) to allow fluid to reach the swellable material 34. The openings 36 can be distributed in such a way that the sum of the diameters of the openings 36 along the direction of motion of the piston 27 is dependent on the position of the piston 27, such that fluid inflow changes in a controlled manner as the piston 27 moves.

Alternatively, the expanding compartment 28 could be loaded pneumatically or hydraulically with a pump. It would alternatively or additionally be possible to provide a non-pressurized passive diffuse discharge of active ingredient from the active ingredient reservoir 23, or discharge via an electrical potential, for example by an iontophoretic transport. Within the active ingredient reservoir 23, the active ingredient 31 can be present alone or embedded into a (possibly swellable) matrix.

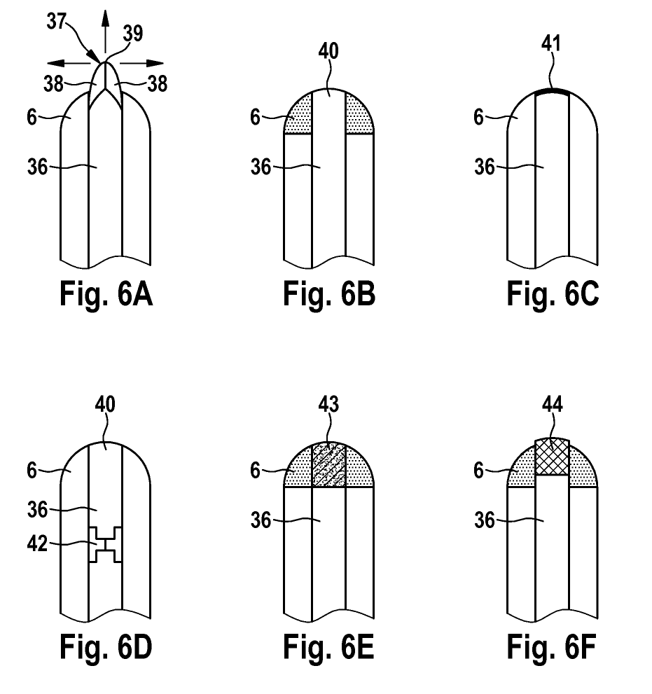

In FIGS. 6A to 6F, schematic views of several variants are illustrated. Here, both the collar 14 (not shown for sake of simplicity) and the tip electrode 6 are designed to discharge at least one active ingredient. The tip electrode 6 is provided with a cavity 36 that serves as an active ingredient reservoir, and which is connected in a fluid-conveying manner to a fluid pump. The active ingredient can be discharged through the fluid pump by the tip electrode 6.

In the variant shown in FIG. 6A, at the distal end of cavity 36, a gasket 37 is located with two sealing lips 38 pre-loaded to close, and which can be pushed apart (in the direction of the arrows) against the loading force by the pressurization of the fluid transported by the fluid pump. This opens a passage 39 for discharge of the active ingredient to the surrounding area.

In the variant shown in FIG. 6B, the cavity 36 is provided with an open distal end 40, so that the fluid active ingredient can be discharged to the surrounding area upon the application of corresponding pressurization by the fluid pump.

In the variant shown in FIG. 6C, the distal end of cavity 36 is provided with a membrane 41 so that upon pressurization of the fluid pump, the active ingredient can be discharged to the surrounding area. The membrane 41 can (for example) be a porous membrane, or a closed membrane that allows diffusion of active ingredient.

In the variant shown in FIG. 6D, a gasket 42 similar to the lips 38 of FIG. 6A is mounted at a distance from the distal end of cavity 36. Upon pressurization of the fluid transported by the fluid pump, the gasket 42 may open in order to discharge active ingredient through the open distal end 40 of cavity 36.

In the variant shown in FIG. 6E, the distal end of cavity 36 is provided with a stopper 43 of a sintered material that is suitable for discharging active ingredient so that upon pressurization by the fluid pump, an active ingredient can be discharged to the surrounding area.

In the variant shown in FIG. 6F, the distal end of cavity 36 is provided with a cover 44 of wire gauze, so that upon pressurization by the fluid pump, an active ingredient can be discharged to the surrounding area.

In the variants shown in FIGS. 6A to 6F, the discharge of active ingredient can be controlled by a fluid pump housed in the coronary pacemaker. Such a fluid pump can be supplied by the power supply of the coronary pacemaker, and controlled by the electronic control unit of the coronary pacemaker. In such an arrangement, the discharge of active ingredient can be adjusted to the current status of the inflammation. It is also possible to discharge several active ingredients for which a number of cavities 36 might be provided in the tip electrode 6. It would also be possible to provide one or more reservoirs for active ingredient in the coronary pacemaker that are connected to at least one fluid-conveying cavity 36. While the variants shown in FIGS. 6A to 6F are described in connection with the tip electrode 6, these can equally be incorporated into a screw-in electrode 19.

The active ingredient used in the invention is preferably selected from the following classes of medications: antimicrobial, antimitotic, antimyotic, antineoplastic, antiphlogistic, antiproliferative, antithrombotic and vasodilatory substances.

Especially preferred active ingredients are triclosan, cephalosporin, aminoglycoside, nitrofurantoin, penicillins such as dicloxacillin, oxacillin as well as sulfonamide, metronidazol, 5-fluoruracil, cisplatin, vinblastine, vincristine, epothilone, endostatin, verapamil, statins such as mevastatin, cerivastatin, atorvastatin, simvastatin, fluvastatin, pitavastatin, pravastatin, rosuvastatin as well as lovastatin, angiostatin, angiopeptin, taxane as well as paclitaxel, immunosuppressives or immunomodulators as well as, for example, rapamycin or its derivatives such as biolimus, everolimus, deforloimus, novolimus, methotrexat, colchicine, flavopiridol, suramin, cyclosporin A, clotrimazole, flucytosin, griseofulvin, ketoconazole, miconazole, nystatin, terbinafine, steroides, sulfasalazine, heparin and its derivatives, urokinase, ppac, argatroban, aspirin, abciximab, synthetic antithrombin, bivalirudin, enoxoparin, hirudin, r-hirudin, protamine, prourokinase, streptokinase, warfarin, flavonoids such as 7,3′,4′-trimethoxyflavone, sartane as well as dipyramidol, trapidil, and nitroprusside.

The active ingredients can be used individually, or combined in equal or various concentrations.

As has been explained above in regards to the exemplary versions, the invention provides perforation protection via a radially broadened structure in the distal end section of the probe body adjacent the electrode that is mounted at the distal end of the electrode probe. Therapeutically efficient, local discharge of active ingredients at the site of tissue irritation can be achieved by the discharge of active ingredients via the radially broadened structures, and/or via the electrode mounted at the distal end of the electrode probe. Particularly when a screw-in electrode is used, traumatization of the coronary tissue can occur as a result of the insertion of the screw-in electrode, particularly at the tissue surrounding the implant. As a result of a discharge of anti-inflammatory active ingredients by the screw-in electrode, the active ingredients are placed at the target location. In addition to faster application, higher concentrations of active ingredients can be achieved in the target tissue, as the diffusion from the endocardium into the myocardium takes place on a delayed basis. Extraction of the released active ingredient by the circulation of blood in the heart is also prevented. Extraction of the released active ingredients tend to be most pronounced in the first days after the implantation, as the tip of the electrode is still exposed or not yet grown in. At the same time, however, inflammation is at its peak.

It will be apparent to those skilled in the art that numerous modifications and variations of the described examples and versions are possible in light of the foregoing discussion. The disclosed examples and versions are presented for purposes of illustration only. Therefore, it is the intent to cover all modifications and alternate versions that are literally or equivalently encompassed by the following claims.

What is claimed is:

1. An electrode probe for medical use including:
a. an elongated flexible probe body extending from a proximal end to a distal end, the distal end including a distal end section which is at least partially transformable between a narrow condition and a radially broadened condition,
b. an electrode mounted on the distal end section,
c. an electrical supply line extending within the probe body to the electrode,
d. a displacement mechanism coupled to the distal end section, wherein actuation of the displacement mechanism transforms the distal end section between the narrow condition and the radially broadened condition.

2. The electrode probe of claim 1 wherein the displacement mechanism:
a. displaces the electrode and the proximal end of the probe body toward each other when transforming the distal end section into the radially broadened condition, and
b. displaces the electrode and the proximal end of the probe body away from each other when transforming the distal end section into the narrow condition.

3. The electrode probe of claim 1 wherein:
a. the displacement mechanism includes a casing surrounding at least a portion of the probe body, and
b. relative motion between the casing and the electrical supply line transforms the distal end section between the narrow condition and the radially broadened condition.

4. The electrode probe of claim 1 wherein:
a. the electrode is:
(1) helical, and
(2) displaceable into and out of the probe body by rotating the electrical supply line, and
b. displacement of the electrode into and out of the probe body also transforms the distal end section between the narrow condition and the radially broadened condition.

5. The electrode probe of claim 4 further including a thrust bearing within the probe body at or near the distal end, wherein the electrode bears against the thrust bearing during the electrode's displacement to transform the distal end section between the narrow condition and the radially broadened condition.

6. The electrode probe of claim 1 wherein at least a portion of the distal end section includes one or more lamellar strips having:
a. lengths extending at least substantially in an axial direction with respect to the probe body, and
b. opposing sides extending between opposing slots in the probe body.

7. The electrode probe of claim 1 wherein:
a. the distal end section includes extensions displaceable between:
(1) the narrow condition, wherein the extensions extend in abutment with the outer circumference of the probe body, and
(2) the radially broadened condition, wherein the extensions extend radially outwardly from the outer circumference of the probe body; and
b. the extensions are biased toward the radially broadened condition.

8. The electrode probe of claim 1 wherein at least a portion of an exterior surface of the distal end section includes at least one active ingredient thereon for discharge into body tissue.

9. The electrode probe of claim 1 wherein the distal end section includes:
a. an active ingredient reservoir, and
b. a discharge mechanism configured to discharge at least one active ingredient from the reservoir into body tissue.

10. The electrode probe of claim 9 wherein the active ingredient reservoir is situated within the probe body.

11. The electrode probe of claim 9 further including a pump situated between the ingredient reservoir and the discharge mechanism, wherein the pump supplies active ingredient from the reservoir to the discharge mechanism.

12. The electrode probe of claim 1 wherein the electrode includes a passage therein, the passage being connected to an active ingredient reservoir for discharging at least one active ingredient into body tissue.

13. The electrode probe of claim 12 further including a piston displaceable to compress the active ingredient reservoir.

14. The electrode probe of claim 13 further including swellable material adjacent the piston, wherein swelling of the material displaces the piston.

15. The electrode probe of claim 14 wherein the swellable material is contained within a compartment in the electrode probe, the compartment having a wall having one or more liquid-permeable openings therein.

16. The electrode probe of claim 15 wherein the area of the liquid-permeable openings changes as the piston is displaced.

17. An electrode probe for medical use including:
a. an elongated probe body extending between a proximal end and a distal end, the probe body having:
(1) a distal end section extending proximally from the distal end,
(2) a sleeve situated proximally of the distal end section,
b. an electrode exposed on the distal end,
c. an electrical supply line extending proximally from the electrode within the probe body,
wherein displacement of at least one of the distal end section and the sleeve toward the other causes at least a portion of the distal end section to extend radially outwardly to define an at least substantially planar tissue contact surface from which the electrode extends.

18. The electrode probe of claim 17 wherein at least a portion of the tissue contact surface bears active ingredient.

19. An electrode probe for medical use including:
a. an elongated probe body having an axial length extending between a proximal end and a distal end, the probe body having an outer circumference including:
(1) a distal end section which is at least partially transformable between a narrow condition and a circumferentially broadened condition,
(2) a sleeve situated proximally of the distal end section,
b. an electrode mounted on the distal end section,
c. an electrical supply line extending within the sleeve to the electrode, wherein:
i. displacement of at least one of the electrode and the sleeve toward the other causes at least a portion of the distal end section to axially narrow and circumferentially broaden, and
ii. displacement of at least one of the electrode and the sleeve away from the other causes at least a portion of the distal end section to axially lengthen and circumferentially narrow.

20. The electrode probe of claim 19 wherein at least a portion of the distal end section bears active ingredient.
